(19) United States
(12) Patent Application Publication
(10) Pub. No.: US 2013/0317315 A1
(43) Pub. Date: Nov. 28, 2013

(54) METHOD OF AGE MANAGEMENT

(71) Applicants: Tony V. Lu, Bellevue, WA (US); Bruce Chau, Berkley, MI (US); Thomas Tsang, Brooklyn, NY (US)

(72) Inventors: Tony V. Lu, Bellevue, WA (US); Bruce Chau, Berkley, MI (US); Thomas Tsang, Brooklyn, NY (US)

(21) Appl. No.: 13/900,291

(22) Filed: May 22, 2013

Related U.S. Application Data
(60) Provisional application No. 61/650,092, filed on May 22, 2012.

(51) Int. Cl. A61B 5/00

(52) U.S. Cl. CPC A61B 5/48
USPC 600/300; 604/507; 604/542

(57) ABSTRACT

A method of age management, by consulting with a patient, performing comprehensive testing on the patient, determining the physiological, functional, and biological age of the patient, and recommending and performing treatments based on the physiological, functional, and biological age of the patient. The performing comprehensive testing is further defined as performing a test of standard plus functional medicine testing, complete hormonal panel, infectious disease panel, tumor markers panel, and combinations thereof.

### BACKGROUND OF THE INVENTION

1. Technical Field

The present invention relates to methods of age management.

2. Background Art

A large percentage of the world is getting increasingly older. This is especially a problem in developed countries, where low birth rates will greatly increase the number of people over the age of 65. Age management, the practice of extending life and treating age-related diseases, is becoming more relevant as the population ages.

Currently, no age management protocol provides a comprehensive approach to address the aging process from head to toe and from internal to external. For example, a 46 year old menopausal woman with eye bags and difficulty walking up the stairs because of degenerative joint disease in the knees goes to see her doctor for age management. If she has only her eye bags fixed without addressing her knees and hormonal status, her provider is not delivering a complete range of state of the art options and services.

There remains a need for an age management method that addresses every aspect of the aging process and provides rejuvenation for patients.

### SUMMARY OF THE INVENTION

The present invention provides for a method of age management by consulting with a patient, performing comprehensive testing of the patient, determining the physiological, functional, and biological age of the patient, and recommending and performing treatments based on the physiological, functional, and biological age of the patient.

### DETAILED DESCRIPTION OF THE INVENTION

The present invention is generally directed to a method of total age management by consulting with a patient, performing comprehensive testing of the patient, determining the physiological, functional, and biological age of the patient, and recommending and performing treatments based on the physiological, functional, and biological age of the patient.

The method provides a complete solution from head to toe and from internal to external for the inevitable process of degeneration and aging which all men and women have to face after reaching a certain age. Based on the consultation and testing, it can be determined why a patient's body functions like a particular age when the patient is younger (i.e. why the body functions like a person in their fifties or sixties when the person is in fact in their thirties), and specific treatments detailed below can be recommended and performed to improve the function of the body. Similarly, if a patient desires to look, act, and feel younger (i.e. a sixty or seventy year old wants to function as a forty year old), specific treatments detailed below can be recommended and performed to improve the function of the body. The method of analysing and treating the whole body results in a rejuvenated individual because the method is able to maintain the patient's body at peak performance as long as the patient desires. Currently, programs attempt to address some of the issues that the aging population faces, but they are not able to provide management for every aspect or determine the physiological, functional, and biological age and recommend appropriate treatments. The method of the present invention is the most comprehensive approach to manage the aging process through high tech and innovative means.

When a new patient comes in to receive total age management assessment, the following steps are performed. The patient consults with a physician. Comprehensive testing is performed including, but not limited to, standard plus functional medicine testing, complete hormonal panel, infectious disease panel, tumor markers panel, and combinations thereof. Based on the consultation and the test results, the physiological, functional, and biological age of the patient is determined. The following treatments can then be recommended and performed based on the physiological, functional, and biological age of the patient.

Detoxification (Liver, gastrointestinal tract, Heavy Metals if needed) can be performed. IV therapy can be performed to enhance the nutritional and immune status. Hormone restoration can be performed (such as cortisol, DHEA, thyroid hormone, estrogen, progesterone, testosterone, or growth hormone, or combinations thereof). A “FitLipo” procedure (Fat Isolation Technique with Smooth Liposuction and Stromal Vascular Fraction (SVF) Extraction from the patient's own fat) can be performed (i.e. preferably a liposuction procedure that results in a smooth skin surface, especially at the abdomen). Stromal vascular fraction (SVF) IV infusion can be performed with activation for systemic rejuvenation and repair. SVF/PRP (platelet-rich plasma) facial and hands rejuvenation can be performed ((with or without botulinum toxin) to inject into the face and hands to provide a smooth, firm and youthful look; the fat from the liposuction can also be used as fillers; the SVF and PRP helps repair damaged skin). SVF/PRP bone and joint injection can be performed if needed (such as steroids, using SVF and PRP to regenerate cartilage for people with wear and tear injury and osteoarthritis of the joints). SVF scalp injection can be performed with hair restoration if needed (such as by prescription medication, transplant surgery, or bridging hair without surgery). Sexual health enhancement can be performed if needed (such as erectile dysfunction treatments (sildenafil), natural supplements, hormonal restoration and using SVF and PRP for injection into G spot for female patients and genital organ for male patients to rejuvenate the sexual organ and to enhance sexual performance). Any other suitable aesthetic procedures can be performed if needed. All of these steps can be performed, or combinations of the steps can be performed depending on the needs of the patient.

Every aspect of the method can be integrated with a patient's Electronic Health Record (EHR). Preferably, the EHR is cloud-based. A completely new EHR can be created for a patient, or information can be added to an existing EHR. The EHR can keep track of a patient's progress with the age management method. The EHR can contain structured templates to guide the delivery and practice of age-management protocols and other modules if providers and clinics are part of the network. This facilitates a “turn-key” solution for risk-assessment, data capture, and clinical support for patent-pending protocols. Updates to the practice guidelines can be done centrally (and can be annually and can be provided by consultants) and can be provided to any user so that the provider does not need any IT consultants or servers. A patient portal and management system can be set up to provide direct interactions with patients including a scheduling system. A separate learning module and e-learning tool can be created for providers to update skills and knowledge base.

There are several advantages of the method of the present invention. The method ensures that the aging process is addressed from head to toe, from inside out. In addition, this protocol assesses all aspects of aging including the evaluation of other systems that can be degenerating but as yet show no obvious symptoms, provides access and delivery through an innovative cloud based Electronic Health Record (HER) system, and ensures timely and better quality age-management care at the highest efficiency. Detection of degeneration in individuals that do not show obvious symptoms is accomplished by being thorough and comprehensive in the exam of the patient. Because there is a systematic protocol to assess the aging process from head to toe and from inside out, the chance that the provider will miss something or the patient will forget to mention something is miniscule. A set of questionnaires can be provided as well to help the patient recall signs and symptoms that can be specific for certain medical conditions. In addition, all clients undergo a very thorough physical examination by our specially trained physicians. The Electronic Health Record (EHR) can also assist in retrieving and updating all past and present medical conditions that the patient may or may not remember.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A method of age management, including the steps of:
consulting with a patient;
performing comprehensive testing on the patient;
determining the physiological, functional, and biological age of the patient; and
recommending and performing treatments based on the physiological, functional, and biological age of the patient

2. The method of claim 1, wherein said performing comprehensive testing is further defined as performing a test chosen from the group consisting of standard plus functional medicine testing, complete hormonal panel, infectious disease panel, tumor markers panel, and combinations thereof.

3. The method of claim 1, wherein said performing comprehensive testing allows for detection of degeneration in the patient that does not show obvious symptoms.

4. The method of claim 1, wherein said performing comprehensive testing further includes the patient filling out questionnaires about signs and symptoms for medical conditions.

5. The method of claim 1, wherein said recommending and performing step is further defined as performing a detoxification chosen from the group consisting of liver, gastrointestinal tract, heavy metals, and combinations thereof.

6. The method of claim 1, wherein said recommending and performing step is further defined as performing intravenous therapy and enhancing the nutritional and immune status of the patient.

7. The method of claim 1, wherein said recommending and performing step is further defined as performing hormone restoration of a hormone chosen from the group consisting of cortisol, DHEA, thyroid hormone, estrogen, progesterone, testosterone, growth hormone, and combinations thereof.

8. The method of claim 1, wherein said recommending and performing step is further defined as performing a liposuction procedure that results in a smooth skin surface.

9. The method of claim 1, wherein said recommending and performing step is further defined as performing a stromal vascular fraction (SVF) infusion and activating systemic rejuvenation and repair.

10. The method of claim 1, wherein said recommending and performing step is further defined as performing a SVF/PRP (platelet-rich plasma) facial and hands rejuvenation by injecting SVF/PRP into the face and hands.

11. The method of claim 10, further including the step of injecting botulinum toxin into the face and hands.

12. The method of claim 1, wherein said recommending and performing step is further defined as performing SVF/PRP bone and joint injection.

13. The method of claim 12, wherein said performing SVF/PRP bone and joint injection step regenerates cartilage.

14. The method of claim 1, wherein said recommending and performing step is further defined as performing SVF scalp injection and hair restoration by a method chosen from the group consisting of administering prescription medication, transplant surgery, or bridging hair without surgery.

15. The method of claim 1, wherein said recommending and performing step is further defined as performing sexual health enhancement by a method chosen from the group consisting of administering erectile dysfunction treatments, administering natural supplements, providing hormonal restoration, and using SVF and PRP for injection into the G spot for female patients and genital organ for male patients to rejuvenate the sexual organ and to enhance sexual performance.

16. The method of claim 1, further including the step of using the patient's electronic health record and keeping track of the patient's progress with said age management method.
